(19) United States
(12) Patent Application Publication
(10) Pub. No.: US 2009/0192363 A1
(43) Pub. Date: Jul. 30, 2009

(54) METHOD AND SYSTEM FOR DELIVERING CLINICAL LAB QUALITY AND PROFESSIONAL INTERPRETATION TO HOME AND CLINIC TESTING

(76) Inventor: ROBERT CASE, Henderson, NV (US)

Correspondence Address: MINTZ, LEVIN, COHN, FERRIS, GLOVSKY AND POPEO, P.C, ONE FINANCIAL CENTER, BOSTON, MA, 02111, US

(21) Appl. No.: 12/361,420

(22) Filed: Jan. 28, 2009

Related U.S. Application Data
(60) Provisional application No. 61/024,254, filed on Jan. 29, 2008.
(60) Provisional application No. 61/028,269, filed on Feb. 13, 2008.

(51) Int. Cl. A61B 5/00

(52) U.S. Cl. 600/300; 713/193

(57) ABSTRACT

A diagnostic testing system includes a network node comprising a controlling entity communicatively coupled to a network. The controlling entity communicates with a patient, a laboratory facility, and a medical decision maker over the network. The controlling entity controls the transfer of diagnostic data between the patient, the laboratory facility, and the medical decision maker over the network. The diagnostic data may be related to at least one diagnostic test on the patient.

### REFERENCE TO PRIORITY DOCUMENTS

This application claims priority of co-pending U.S. Provisional Patent Application Ser. No. 61/024,254, filed Jan. 29, 2008, U.S. Provisional and Patent Application Ser. 61/028,269, filed Feb. 13, 2008. Priority of the aforementioned filing dates is hereby claimed and the disclosures of each Provisional Patent Applications are hereby incorporated by reference in their entirety.

### BACKGROUND

The present disclosure relates to moderately complex diagnostic testing and, in particular, to systems and methods for performing complex diagnostic testing without requiring the transport of either a sample or a tested party to a clinical laboratory or medical facility location and while also providing a requisite level of system use and quality control.

With an ever expanding list and quality of very simple to use biological tests, there is a growing pressure to provide these tests to the patient to accommodate the desired frequency of testing economically and conveniently. Unfortunately with current devices, medical professionals are dissociated from the process. However, although the patient can generally perform the steps in the test procedure on his or her own, the patient is generally not qualified to verify the proper functioning of the testing system, confirm the efficacy of its quality control mechanisms and fully interpret the validity and meaning of the test results. Even if properly interpreted, communication with the consumer's health provider over an uncontrolled medium compromises the consumer's personal healthcare confidentiality.

In view of the foregoing, there is a need for systems and methods to overcome these shortcomings while providing the convenience, clinical effectiveness and confidentiality of home health monitoring for biological fluids.

### SUMMARY

The disclosed systems and methods preserve the current attributes of convenience and noninvasiveness of in-home testing, and enhances them by requiring licensed clinical laboratory control of the testing system and promoting physician-directed monitoring compliance. The systems and methods of the present invention deliver more accurate testing than conventional systems and methods by bringing clinical laboratory quality and professional interpretation to home testing.

In one aspect, there is disclosed a diagnostic testing system, comprising a network node comprising a controlling entity communicatively coupled to a network. The controlling entity communicates with a patient, a laboratory facility, and a medical decision maker over the network, and controls the transfer of diagnostic data between the patient, the laboratory facility, and the medical decision maker over the network. The diagnostic data may be related to at least one diagnostic test on the patient.

In another aspect, there is disclosed a remote diagnostic testing system, comprising a network node comprising a clinical diagnostic laboratory facility. The laboratory facility receives encoded diagnostic test data from a patient over the network. The clinical diagnostic laboratory facility decodes and processes the result data into a format that is readable by a medical decision maker. The diagnostic laboratory facility transmits the processed result data to the medical decision maker over the network for interpretation by the medical decision maker.

Other features and advantages should be apparent from the following description of various embodiments, which illustrate, by way of example, the principles of the disclosed devices and methods.

### BRIEF DESCRIPTION OF THE DRAWINGS

FIG. 1 shows a schematic representation of an exemplary embodiment of a system for performing diagnostic testing over one or more remote locations.

FIG. 2 shows a schematic representation of exemplary equipment that the patient uses to obtain diagnostic data.

### DETAILED DESCRIPTION

Disclosed is a system for enabling and performing complex diagnostic testing without transporting either a diagnostic sample or a patient to a clinical laboratory or medical facility. The disclosed system permits asynchronous performance of testing steps that maximize efficiency of time required to perform, verify, and complete the diagnostic process while maintaining a high level of quality control. The disclosed system further permits a method of mandatory, real-time, bi-directional and automatic control of the diagnostic testing process outside of the laboratory by laboratory professionals. The system enables on-demand testing by an individual comprised of the patient, physician, and/or medical technician. This establishes availability of results for clinician assessment and intervention, which can be particularly positive for chronic disease testing. As mentioned, the system includes automated verification and quality control of the entire system, the user, and the test itself to assure the patient, laboratory professional, and clinician of the reliability of the remote testing and test results.

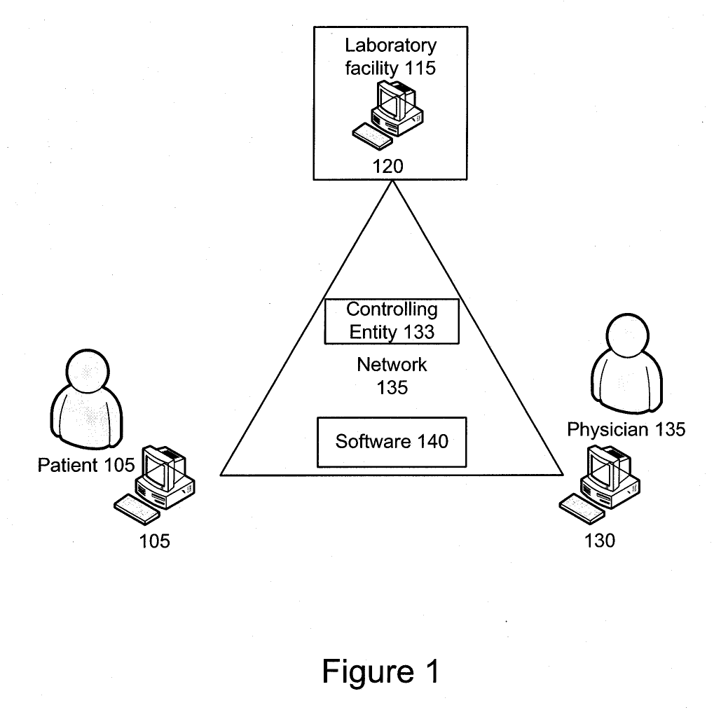

FIG. 1 shows a schematic representation of an exemplary embodiment of a system 100 for performing diagnostic testing over one or more remote locations. The system includes a patient 105 upon which a diagnostic test is performed. The patient has access to a patient computer 110. The system 100 further includes a laboratory facility 115 having access to a lab computer 120. The laboratory facility 115 is a government licensed facility. In an embodiment, the laboratory facility is a clinical laboratory facility. The system 100 also includes a physician 125 or other medical clinician that can interpret and/or analyze diagnostic test results and that has access to a physician computer 130. The physician 125 can be a licensed physician or it can be a medical decision maker that is licensed or otherwise qualified or authorized to interpret and/or analyze diagnostic test results. The patient 105, laboratory facility 115, and physician 125 can be located at any of a variety of locations that are remote from one another.

The computers 110, 120, and 130 are adapted to communicate with one another via one or more communication networks 135. The network 135 can comprise, for example, a common or private bi-directional telecommunications network (e.g., a public switched telephone network (PSTN), cellular network, a cable-based telecommunication network, a LAN, a WAN, a wireless network, or combinations thereof), coupled with or overlayed by a TCP/IP network (e.g., the Internet or an intranet). Each of the computers 110, 120, and 130 can be any type of device that is configured to communicate with the network 135 and to establish communication links with one or more of the other computers over the network 100. The type of computer can device can vary and can include, for example a personal computer, personal digital assistant, land-line telephone, netbook, wireless telephone/smartphone, etc. configured to transmit and receive data over a network.

The system further includes software 140 that manages communication between the computers 110, 120, and 130. The software 140 may reside on the network or may reside in separate components on the individual computers such that the separate components can communicate with one another.

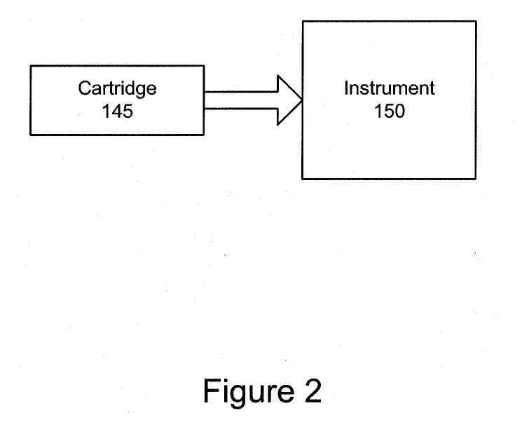

With reference to FIG. 2, the patient 105 has access to at least one test cartridges 145 that is adapted to accept a biological sample from the patient 105. The test cartridge 145 can vary and can include, for example, a cassette, disk, cylinder, or any other device that may be connected to a diagnostic instrument.

The biological sample can vary and can include, for example, blood, saliva, urine, interstitial fluid, plasma, serum, etc.

The cartridge 145 interfaces with a diagnostic instrument 150 that is adapted for testing the sample and provides raw sample detection and quality control data, as well as test, user and individual system identification data. The data generated by the instrument 150 is raw in that it is meaningless until forwarded to and reviewed by the laboratory facility 115 over the network 135 for proper identification, QC validity and results interpretation. This removes the patient from the clinical requirements normally served by trained laboratorians. It also allows communication of the diagnostic data in, not only an encoded form, but also in a form that is not interpretable except with a lab key. Additionally, the data source and reagents used to generate the results can be confirmed by the laboratory. This information is then transmitted to the physician 125 for final clinical application and communication back to the patient 105, as described in more detail below. Segregating the diagnostic process in such a manner removes the need for an office visit, except, if necessary, for follow-up and also streamlines the entire healthcare process while adding greatly to the safety of home or otherwise remote testing at significantly reduced total health care cost including testing cost.

In an embodiment, a controlling entity 133 resides on the network or is otherwise communicatively linked to the network 133. The controlling entity 133 can include equipment to enable network communication, such as, for example, servers, call controllers, database equipment, computer processors, memory, etc. The equipment can include computer-readable medium having computer-readable data that includes instructions for performing the processes described herein. The controlling entity 133 is configured to accept and/or control communications to, from, and between the patient computer 110, lab computer 120, and physician computer 130. In an embodiment the controlling entity 133 is also configured to activate and de-activate equipment located at the patient, laboratory facility, and physician locations, such as activation and de-activation of the instrument 150. In this manner, the controlling entity 133 can exercise various degrees of control over the diagnostic testing process, including control over scheduling of tests, activation and deactivation of the instrument, and transfer and analysis of data related to test results.

In order to provide such services and execute such control, the controlling entity 133 may establish a service contract with at least one of the patient, laboratory facility, or physician. Any of the computers can be equipped with one or more applications provided by the controlling entity 133 (such as the software 140) to enable the processes described herein. In addition, the controlling entity 133 can provide the computers with one or more network addresses that are controlled by the controlling entity 133. This enables the controlling entity 133 to control communications related to the addresses.

In an alternate embodiment, the controlling entity 133 and the laboratory facility or the physician are the same entity or are both controlled by the same entity.

In an initial step, the patient 105 launches an instance of the software 140 on the computer 110. The software 140 is adapted to communicate with the instrument 150 so that data may be exchanged between the software 140, computer 110, and the instrument 150. The instrument 150 is activated (such as by using the software 140) and then receives an initial set of data from the software 140, such as data that verifies that the instrument is authorized to interface with the software 140. The patient 105 can provide identification data to the software 140 via the computer 110. In this regard, the software 140 can include a user interface that prompts the patient 150 for necessary data. The patient 140 may be required to enter the appropriate password into the software 140.

After automated system internal quality control by the software 140, the patient is prompted to obtain a sample via the test cartridge 145. The patient 105 provides a biological sample to the test cartridge 145 and couples the test cartridge 145 to the instrument 150. The instrument 150 then automatically performs actions that read data associated with the biological sample. The instrument 150 also performs one or more diagnostic procedures on the sample and outputs result data to the software 140. As mentioned, the software 140 can reside on the computer 110, on the network 135, or a combination thereof. The software 140 is configured to encrypt the result data and to convert it into a format that can be transferred over the network 135. No test result information is available to the patient 140 directly from the instrument 150, cartridge 145, or system software 140 unless it is provided by the laboratory facility 115 or the physician 125 as described below.

In an embodiment, the patient 140 may be prompted by the software 140 to remove the used test cartridge 145 and repeat the process with a new sample and test cartridge per a system software personalized testing protocol (for example, X-times over the course of a testing day, in consideration of various parameters). The personalized testing protocol may have been previously determined by a physician. Upon completion of the last scheduled test and confirmation by the patient, the system software 140 automatically transmits the test data to the lab computer 115, as described below.

Thus, the software 140 transmits the encrypted test data over the network 135 to the lab computer 120 at the laboratory facility 115. As mentioned, the network 135 can include the Internet and/or any wireless communication means. The lab computer 120 includes an instance of the software 140 that is configured to decrypt or otherwise decode the test data. The computer also performs one or more verification and/or quality control functions regarding the test data. This can include, for example, verifying that the data registers with previously-obtained verification information in a patient database and testing quality control information. Once the test data is verified, the lab computer 120 transmits a verification signal to the patient computer 110 via the network 135. Any of the communications between patient, laboratory facility, and physician can be transmitted in a variety of manners, including text message.

The laboratory facility 115 can have access to local and/or remote instrumentation that enhances or otherwise facilitates analysis of the test data. Such instrumentation can include, for example, scales, thermometers, or imaging systems. The laboratory facility or other facility can also provide interactive user training and operational instruction media (audio/video, text, diagrams, etc.) to the patient computer 110 to support proper operation of the system by the patient 105.

Additionally upon the lab computer's verification of the test data, the lab computer (via the software 140), generates the specific test results and an electronic test results report for verification at the laboratory facility. The verification can be performed automatically by the lab computer 120 or can involve human verification. Thus, where legally allowable, the laboratory facility 120 may use “auto-verification” software to verify and release the test results without the direct involvement of an accredited lab technician. The test results report, in an electronic format, is then encrypted by the lab computer 120 and is transmitted to the physician computer 130 over the network 135. The lab computer 120 can also send a verification signal to the patient computer 110 that the test results report has been sent to the physician computer 130. In this manner, the patient 105 can be kept apprised of the process. It is noted that at this stage the lab computer 120 only sends a verification signal to the patient 110 and does not send any data that describes the results.

Upon receipt of the electronic test results, the physician 125 can review and also clinically interpret the results. The physician 125 can use the physician computer 135 to send corresponding information (such as, for example, instructions, interpretations, comments, etc.) to the patient computer 110 via the network 135. The physician can send such information in an encrypted format that requires the software 140 to decrypt. Also, the software 140 on the physician computer 130 can perform a verification step that verifies that the result data corresponds to the patient 105 and also verifies that the physician indeed should be able to access the result data. In another embodiment, the physician computer sends instructions to the lab computer to send the result data to the patient directly from the laboratory facility after the physician has reviewed and interpreted the result data.

Any of the communications between the computers in the above-described process can include one or more “handshake” communications to, for example, assure the patient 105 that relevant data has been received and that the data passes quality control requirements. The communications can also be used to provide a reminder or to alert the patient 105, such as to remind him or her of the need to take a sample, perform a test, etc. and to alert the physician 125 of an immediately pending result. This communication can be done by multiple means, including text messaging for example. Additionally the actual generation of the any of the raw diagnostic data can be generated at a remote professional location such as a clinic staffed by nurse practitioners or the equivalent and then sent to the laboratory facility 115 for secure, higher level analysis and interpretation prior to release to the patient 105. This provides access to a wider variety and more difficult to interpret tests.

Any of the components of the system can utilize any of a variety of encryption technologies, including types that are specific to a single user, further enhancing patient privacy and security.

In an embodiment, the system 100 can be at least partially accessed via a World Wide Web based protocol such as by accessing one or more linked websites that coordinate referral and access of the system 100. In a non-limiting example, a patient may seek anti-aging, disease monitoring, environmental/biohazard/food testing, hormone balance and treatment information, wellness education and testing/monitoring resources. These are just examples of the type of information that the patient may seek. The patient may be directed (such as via sponsored web links, direct marketing, media, partner physicians, pharmacists, wellness clinics and other sources) to a partner website that is run by a controlling entity that controls the system 100 or by a partner to the controlling entity.

In an exemplary process, a consumer (e.g., the patient 105) logs onto the partner website, which provides access to, for example, educational materials, references and links to related information, including a link to the controlling entity's website. Prior to or during the patient's access to the controlling entity's website, the patient may be requested to provide information, such as basic, non-financial, personal information. This may qualify the patient for a free, statically-based hormone “balance” assessment. As mentioned, this is just an example and the patient may qualify for other types of assessments or diagnostic procedures. The patient can then provide, via the website, individual readily-available wellness data and immediately receive the assessment via the website.

The website may also direct the patient to one or more partner physicians or the patient can enlist his or her personal physician for the hormone testing/monitoring recommendations and treatment options. In an embodiment, concurrent with the results of the free assessment, the website may present the patient with one or more subscription membership packages, products and services and related personalized wellness information based on the free assessment results and other consumer-provided data. Such packages can include access to the remote diagnostic system 100 described above. The patient can chooses to subscribe to any of a variety of personalized program package (such as an annual wellness monitoring and evaluation program package) and potentially other services to be determined in conjunction with the partner website. In this regard, the patient may be requested to provide finance data, such as credit card information. In a non-limiting embodiment, the personalized program package relates to OTC saliva-based hormone replacement/balance monitoring for men and women, including such tests as Estrogen, Progesterone, Testosterone, Cortisol, DHEA and Androstenedione. Other programs may include, for example, diagnostic testing and monitoring for sexual dysfunction, infertility, and long-term disease state management (i.e., diabetes), among others.

Upon the patient enrolling into a program, the website directs the patient to a partner physician for wellness guidance, consultation and/or treatment in conjunction with the purchased monitoring program. Alternately, the patient may enlist his or her current physician for ongoing testing/monitoring recommendations and treatment options. In addition, the controlling entity establishes a unique, secure account for the patient that includes access to the system 100. After establishment of the patient's account, the controlling entity processes and ships to the patient a personalized order for the selected monitoring program. The order fulfillment may include testing equipment and user information, which is desirably packaged in a personally and uniquely identified subscriber monitoring test kit. The kit may include:

(1) a supply of personally and uniquely identified single-use, self-contained disposable test cartridges 145. The cartridges may be room temperature stable and sufficient in quantity for the pre-payment period;

(2) a supply of sample collection devices designed to deliver the collected sample directly and accurately into the test cartridge 145;

(3) a personally and uniquely identified test cartridge instrument 150. The instrument may include plug-and-play connectivity and embedded software (such as an instance of the software 140) for generating test data, automated communication with the subscriber's home computer and bi-directional system function, user operation and test protocol QC; and

(4) a version of the software 140 that can be loaded onto the patient computer 110 for secure automated connectivity with the laboratory facility 115, a website server of the controlling entity, and a individual/physician monitoring compliance support and test cartridge reader.

The patient is also provided with secure unique subscriber account on the controlling entity's website. A unique account is established for the patient's designated physician. In coordination with the physician, the software 140 is downloaded and installed on the physician computer 130.

Once the software 140 is installed on the patient's computer 110, the controlling entity verifies connectivity and performs security and test system quality control checks. The software 140 reminds and directs the patient to perform the required periodic quality control checks before running actual tests. The system provides quality control operator support to correct any QC issues and/or automatically connects with customer support for additional guidance/procedures.

The software 140 may send periodic reminders to the patient as to a scheduled testing regimen and compliance requirements with their physician's instructions, regarding tests, testing days and sampling/multiple test requirements. In this regard, the patient may be assessed by the physician and a clinical monitoring regimen may determined. The physician uses the software 140 supplied to their office to create a guide for the patient for the monitoring regimen at to be performed at home. As discussed above, the patient is provided with the appropriate home testing system for the specific test for their monitoring requirements. This includes testing instrument, computer software and associated single-use disposable or reusable test cartridges. Different versions of the system are capable of performing a variety of analytical methodologies, including, but not limited to immunoassays, hematology, electrolytes, chemistry and molecular tests, among others.

The software 140 as well as the computers 110, 120, and 130, the instrument 150 can collectively work as a system to provide test procedure steps, sampling checklist and QC procedures. The testing cycle guidance may include diet, time of day, exercise and other parameters to ensure the highest quality sample collection protocol and test result quality. Upon expiration or cancellation of the consumer's account, the consumer's unique home computer software 140 and instrument 150 are blocked from connecting with the lab computer 115 and physician computer 125. In an embodiment, the consumer buys one or more components of the system, such as the cartridge 145 or the instrument 150, at a retail outlet such as a pharmacy, medical supply store, health product store, etc. The component(s) may include software or hardware that enables the components to be controlled and/or monitored by the software 140.

As will be apparent to those of skill in the art upon reading this disclosure, each of the individual embodiments described and illustrated herein has discrete components and features which may be readily separated from or combined with the features of any of the other several embodiments without departing from the scope of the subject matter described herein. Any recited method can be carried out in the order of events recited or in any other order which is logically possible.

Although embodiments of various methods and devices are described herein in detail with reference to certain versions, it should be appreciated that other versions, embodiments, methods of use, and combinations thereof are also possible. Therefore the spirit and scope of the appended claims should not be limited to the description of the embodiments contained herein.

1. A diagnostic testing system, comprising:
a network node comprising a controlling entity communicatively coupled to a network, wherein the controlling entity communicates with a patient, a laboratory facility, and a medical decision maker over the network, and wherein the controlling entity controls the transfer of diagnostic data between the patient, the laboratory facility, and the medical decision maker over the network, the diagnostic data being related to at least one diagnostic test on the patient.

2. A diagnostic system as in claim 1, wherein the diagnostic data includes results of a diagnostic test on the patient, and wherein the controlling entity requires that at least one clinical laboratory verify and interpret the diagnostic data prior to transfer of the results to the patient over the network.

3. A diagnostic system as in claim 1, wherein the controlling entity utilizes real-time, bi-directional communication over the network to control a remote diagnostic testing device of the patient.

4. A diagnostic system as in claim 1, wherein the controlling entity prompts at least one of the patient, laboratory facility, and medical decision maker over the network regarding patient compliance with a test protocol.

5. A diagnostic system as in claim 1, wherein the controlling entity prevents transfer of the diagnostic data unless predetermined criteria is satisfied.

6. A diagnostic system as in claim 5, wherein the predetermined criteria comprises review of the diagnostic data by a medical decision maker.

7. A diagnostic system as in claim 1, wherein the diagnostic data includes results of a diagnostic test on the patient and wherein the controlling entity ensures that the results cannot be viewed by the patient unless the results are first processed by the clinical laboratory.

8. A diagnostic system as in claim 1, wherein the controlling entity implements a quality control procedure that governs transfer of the diagnostic data over the network.

9. A diagnostic system as in claim 1, wherein the controlling entity implements a component control procedure that controls the use of equipment that produces or interprets the diagnostic data.

10. A system as in claim 1, wherein the diagnostic data relates to at least one of blood, saliva, urine, interstitial fluid, plasma, and serum.

11. A system as in claim 1, wherein the medical decision maker is a physician.

12. A remote diagnostic testing system, comprising:
a network node comprising a clinical diagnostic laboratory facility, wherein the laboratory facility receives encoded diagnostic test data from a patient over the network, and wherein the clinical diagnostic laboratory facility decodes and processes the result data into a format that is readable by a medical decision maker, and wherein the diagnostic laboratory facility transmits the processed result data to the medical decision maker over the network for interpretation by the medical decision maker.

13. A system as in claim 12, further comprising a network node comprising a patient computer that is coupled to the diagnostic laboratory facility over the network, wherein the patient computer interfaces with a test cartridge and a diagnostic instrument that test a sample from the test cartridge to provide the diagnostic test data in an encoded form.

14. A system as in claim 12, further comprising a network node comprising a medical decision maker computer that receives the processed result data from the laboratory facility over the network.

15. A system as in claim 12, wherein the diagnostic laboratory facility sends a verification signal to the patient over the network upon successful transmission of the processed result data to the medical decision maker.

16. A system as in claim 12, wherein the diagnostic laboratory facility is a licensed facility.

17. A system as in claim 12, wherein the encoded diagnostic test data is not readable by the patient.

18. A system as in claim 12, wherein at least a portion of the network is a wireless network.

19. A system as in claim 12, wherein the diagnostic result data relates to at least one of blood, saliva, urine, interstitial fluid, plasma, and serum.

20. A system as in claim 12, further comprising software that controls communication between the diagnostic laboratory facility, patient, and medical decision maker.

21. A system as in claim 12, wherein the medical decision maker is a physician.
